(19) United States
(12) Patent Application Publication
(10) Pub. No.: US 2015/0238217 A1
(43) Pub. Date: Aug. 27, 2015

(54) OPERATION INSTRUMENT

(71) Applicant: SEIKO EPSON CORPORATION, Tokyo (JP)

(72) Inventor: Kazuaki UCHIDA, Fujimi-machi (JP)

(21) Appl. No.: 14/631,693

(22) Filed: Feb. 25, 2015

(30) Foreign Application Priority Data
Feb. 26, 2014 (JP) 2014-035251

(51) Int. Cl. A61B 17/3203; A61B 17/3209

(52) U.S. Cl. CPC A61B 17/3203; A61B 17/3209

(57) ABSTRACT

An operation instrument includes an electric surgical knife, a liquid ejection surgical knife capable of ejecting liquid; and an outer case configured to accommodate the electric surgical knife and the liquid ejection surgical knife, and including an opening which allows an electrode portion of the electric surgical knife to project therefrom. The liquid ejection surgical knife is operated so as to be switched between a therapy mode in which liquid is ejected to the outside of the outer case, and a cleaning mode configured to eject the liquid toward a high-frequency treatment electrode of the electric surgical knife in a state of being accommodated in the outer case.

This patent application claims the benefit of Japanese patent application No. 2014-035251, filed on Feb. 26, 2014. The content of the aforementioned application is incorporated herein by reference in its entirety.

### BACKGROUND

1. Technical Field

The present invention relates to an operation instrument.

2. Related Art

Known examples of an operation instrument include a liquid jet surgical knife configured to perform incision, excision, and the like of living tissues by ejecting liquid (for example, water) continuously at a high speed. As an operation instrument, an electric surgical knife is also known. According to an operation instrument disclosed in JP-T-2009-533109, a structure having both a liquid jet surgical knife and an electric surgical knife in one unit is described. With this structure, a trouble of changing the operation instrument to another instrument during an operation may be eliminated.

The electric surgical knife may cause a living tissue to be stuck to a distal end of the electric surgical knife and affect an accurate technique at the time of usage. Therefore, the electric surgical knife needs to be cleaned every time before using the electric surgical knife. In the related art, it is difficult to clean the electric surgical knife every time before using the electric surgical knife and, even though the trouble of changing the operation instrument to another instrument during the operation may be eliminated as described above, and efficiency of the operation cannot be improved sufficiently.

In addition, in the operation instrument of the related art, a reduction in size, a reduction in cost, resource saving, ease of manufacture, improvement of user-friendliness are desired.

### SUMMARY

An advantage of some aspects of the invention is to solve at least a part of the problems described above, and the invention can be implemented as the following aspects.

(1) An aspect of the invention provides an operation instrument. The operation instrument includes an electric surgical knife, a fluid ejection surgical knife capable of ejecting fluid, and an outer case configured to accommodate the electric surgical knife and the fluid ejection surgical knife, and including an opening which allows an electrode portion of the electric surgical knife to project therefrom. The fluid ejection surgical knife is operated so as to be switched between a first mode in which fluid is ejected to the outside the outer case, and a second mode configured to eject the fluid toward the electrode portion of the electric surgical knife in a state of being accommodated in the outer case. According to the operation instrument of this aspect, the fluid may be ejected to a living tissue by the fluid ejection surgical knife in the first mode, and the fluid may be ejected toward the electrode portion of the electric surgical knife in the second mode. As a result, cleaning of the electrode portion of the electric surgical knife is easily achieved in the second mode. Therefore, an operator's labor for cleaning of the electric surgical knife may be reduced, and hence switching between the electric surgical knife and the fluid ejection surgical knife may be freely performed. Consequently, the operation instrument of this mode achieves an advantage that efficiency of operations is sufficiently improved.

(2) The operation instrument of the aspect described above may be configured such that the fluid ejection surgical knife includes a pulse generating unit configured to generate pulsation in fluid, an ejection tube communicating with the pulse generating unit, and an outer tube in which the ejection tube is inserted, the ejection tube includes a first ejection opening formed at a distal end portion of the ejection tube, and a second ejection opening formed in the ejection tube at a position opposing the electrode portion of the electric surgical knife, the outer tube includes a hole portion at a position capable of aligning with the second ejection opening, and the second ejection opening and the hole portion communicate with each other by moving a relative position between the ejection tube and the outer tube. According to the operation instrument of this aspect, since the first mode and the second mode may be switched only by moving the relative position between the ejection tube and the outer tube, the structure may be simplified.

(3) In the operation instrument of the mode described above, the fluid ejection surgical knife may be configured such that the second ejection opening and the hole portion are brought into communication with each other by projecting the distal end portion of the ejection tube from the outer case in the first mode and retracting the ejection tube into the inside of the outer case in the second mode. According to the operation instrument of this aspect, switching between the first mode and the second mode is achieved in conjunction with switching the movement of the ejection tube between forward and backward.

(4) A second aspect of the invention provides an operation instrument. The operation instrument includes an electric surgical knife, a fluid ejection surgical knife capable of ejecting fluid, and an outer case configured to accommodate the electric surgical knife and the fluid ejection surgical knife, and including an opening which allows an electrode portion of the electric surgical knife to project therefrom. In a fluid passage of the fluid ejection surgical knife is provided with an opening from which the fluid can be ejected toward the electrode portion of the electric surgical knife in a state in which the electric surgical knife and the fluid ejection surgical knife are accommodated in the outer case. According to the operation instrument of this mode, an operator's labor for cleaning of the electric surgical knife is reduced, and hence switching between the electric surgical knife and the fluid ejection surgical knife may be freely performed. Consequently, the operation instrument of this mode achieves an advantage that efficiency of operations may be sufficiently improved.

Not all of a plurality of the components included in respective aspects of the invention are necessarily essential. In order to solve a part of or the entire problems described above or in order to achieve a part of or the entire advantages described in this specification, a part of the plurality of components may be modified, eliminated, or replaced by other components, and a part of limiting contents may be eliminated as needed. In order to solve a part of or the entire problems described above or in order to achieve a part of or the entire advantages described in this specification, a part of or the entire technical characteristics included in one of the aspects of the invention described above may be combined with a part of or the entire technical characteristics included in another aspect of the invention to form an independent aspect of the invention.

For example, an aspect of the invention may be achieved as an apparatus provided with one or more elements out of three members, namely, the electric surgical knife, the fluid ejection surgical knife, and the outer case. In other words, the apparatus may and may not include the electric surgical knife. The apparatus may and may not include the fluid ejection surgical knife. The apparatus may and may not include the outer case. The electric surgical knife may be configured as an electric surgical knife having an electrode portion, for example. The fluid ejection surgical knife may be configured as a fluid ejection surgical knife capable of ejecting fluid, for example. The outer case may be configured as an outer case configured to accommodate an electric surgical knife and a fluid ejection surgical knife, and including an opening which allows an electrode portion of an electric surgical knife to project therefrom, for example. The fluid ejection surgical knife may be configured further as a fluid ejection surgical knife being operated so as to be switched between a first mode in which fluid is ejected to the outside of the outer case and a second mode in which the fluid is ejected toward the electrode portion of the electric surgical knife in a state of being accommodated in the outer case. The apparatus described above may be achieved as the operation instrument for example, and may be achieved as apparatuses other than the operation instrument. In this configuration, at least one of various problems such as a reduction in size, a reduction in cost, resource saving, ease of manufacture, and improvement of user-friendliness of the apparatus may be resolved. Apart of and the entire technical characteristics of the respective aspects of the operation instrument described above may be applied to the apparatus.

The invention may be implemented as various forms other than the apparatus. For example, the invention may be implemented in the form of a method of controlling the operation instrument and the like.

### BRIEF DESCRIPTION OF THE DRAWINGS

The invention will be described with reference to the accompanying drawings, wherein like numbers reference like elements.

FIG. 1 is an explanatory drawing illustrating a configuration of an operation instrument according to an embodiment of the invention.

FIG. 2 is a schematic drawing schematically illustrating a structure of a handpiece.

FIG. 3 is a perspective view of a portion in the vicinity of an opening of an outer case illustrating together with a distal end portion of a liquid ejection surgical knife and a distal end portion of an electric surgical knife.

FIGS. 4A and 4B are explanatory drawings for comparing operations of the liquid ejection surgical knife in a therapy mode and in a cleaning mode.

FIG. 5 is a flowchart illustrating a drive routine of the liquid ejection surgical knife and the electric surgical knife.

FIG. 6 is a flowchart illustrating an electric surgical knife cleaning routine.

### DESCRIPTION OF EXEMPLARY EMBODIMENTS

Hereinafter, embodiments of the invention will be described in the order to an embodiment and modifications.

### A. Embodiment

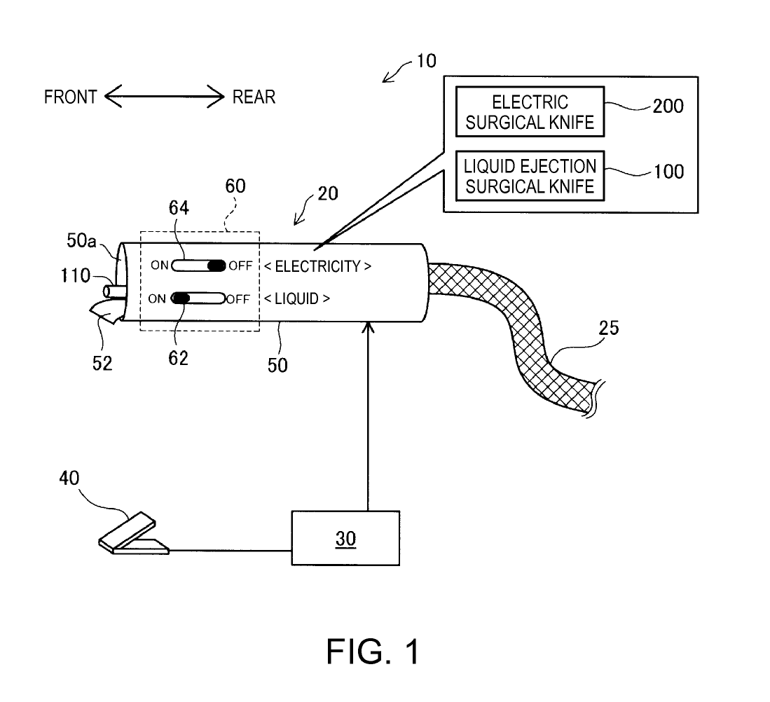

FIG. 1 is an explanatory drawing illustrating a configuration of an operation instrument 10 according to the embodiment of the invention. The operation instrument 10 of this embodiment is an instrument for operations used in medical institutions, and has a function as a surgical knife for incising or excising an affected area.

The operation instrument 10 includes a handpiece 20 and a control unit 30. The handpiece 20 includes a cylindrical-shaped outer case 50, and a liquid ejection surgical knife 100 and an electric surgical knife are accommodated in the interior of the outer case 50.

The liquid ejection surgical knife 100 is used for injecting liquid and performing incision or excision of the affected area with a liquid flow. The electric surgical knife 200 is used for performing incision or clotting (stopping blood) of the affected area by a thermal action of a high-frequency current.

The handpiece 20 is provided with an operating portion 60 on a surface of the outer case 50. The operating portion 60 includes two sliders 62 and 64. A user of the operation instrument 10 is allowed to select a surgical knife to be used from one of two surgical knives 100 and 200 accommodated in the handpiece 20 by selecting and operating one of the two sliders 62 and 64. The slider 62 corresponds to the liquid ejection surgical knife 100, and the slider 64 corresponds to the electric surgical knife 200. When using the respective surgical knives, the user turns the slider corresponding to the surgical knife to be used ON. When one of the sliders is ON, the other slider is OFF. In FIG. 1, the left side in the drawing corresponds to the front side of the handpiece 20, and the right side in the drawing corresponds to the rear side of the handpiece 20. Hereinafter, the front side of the handpiece 20 is simply referred to as “front side” and the rear side of the hand piece 20 is referred to as “rear side”.

If the user selects and operates the slider 62 or 64, a distal end portion of the surgical knife 100 or 200 corresponding to the selected slider 62 or 64 projects from an opening 50a formed on the front side of the outer case 50. The user brings the distal end portion of the surgical knife projecting from the opening 50a close to or into contact with an affected area, and performs incision, excision, or arrest of bleeding from the affected area.

A linear member group 25 extends from an end portion rearward of the outer case 50. The linear member group 25 is a bundle of piping lines and electric wiring lines required for the operations of the two surgical knives 100 and 200.

The control unit 30 is connected to the liquid ejection surgical knife 100 and the electric surgical knife. The control unit 30 controls the operations of the surgical knives 100 and 200. Afoot switch 40 is connected to the control unit 30. If the user turns the foot switch 40 ON and OFF, the surgical knife selected by the user by using the operating portion 60 is driven.

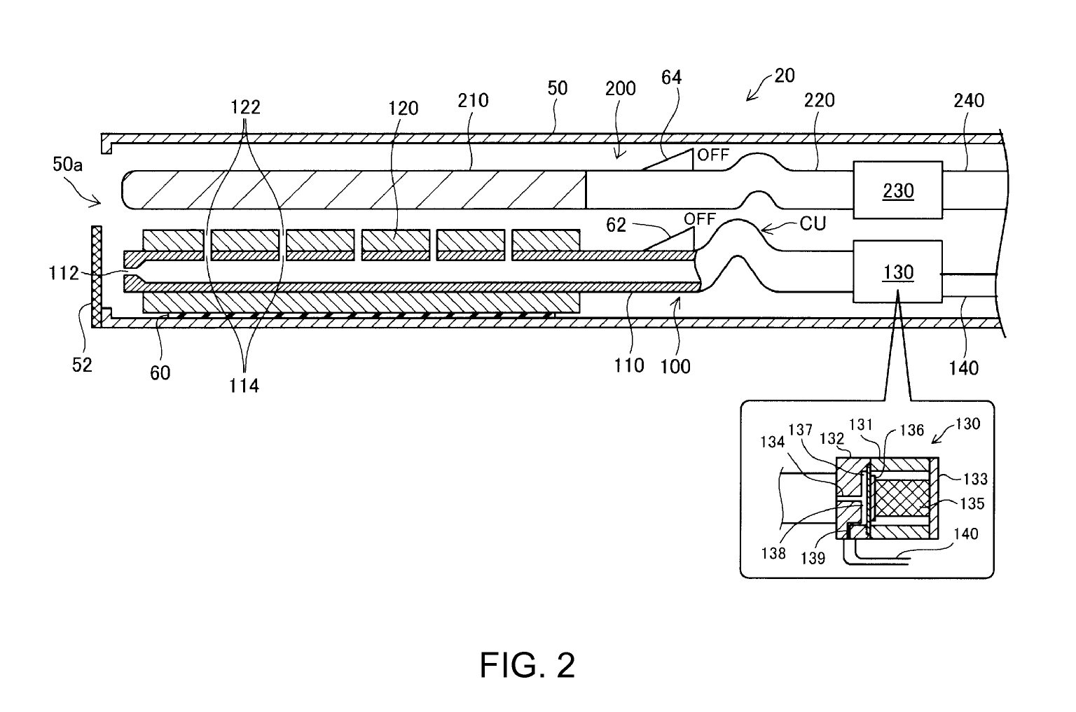

FIG. 2 is a schematic drawing schematically illustrating a structure of the handpiece 20. As described above, the handpiece 20 accommodates the liquid ejection surgical knife 100 and the electric surgical knife.

The liquid ejection surgical knife 100 includes a liquid ejection tube 110, an outer tube 120, an actuator 130, and a liquid supply flow channel 140. The liquid supply flow channel 140 is a flow channel for supplying liquid pumped from a pump (not illustrated) provided outside the handpiece 20 to the actuator 130. The liquid supply flow channel 140 is formed of a flexible member. In this example, the liquid supply flow channel 140 is formed of a PEEK member. The liquid supply flow channel 140 may be replaced by various types of flexible members such as polyvinyl chloride, silicon, or thermoplastic elastomer. As liquid to be supplied to the actuator 130, various types of liquid such as aseptic water for medical use and physiological saline may be employed.

The actuator 130 provides liquid supplied from the liquid supply flow channel 140 with pulsation. The liquid provided with pulsation is supplied to the liquid ejection tube 110, and is ejected as pulsed liquid from an ejection opening 112 formed at a distal end of the liquid ejection tube 110. The term “pulsed liquid” means liquid in a state in which the flow rate or the flow velocity varies. A mode of ejecting the liquid in a pulsed manner includes an intermittent ejection that ejects the liquid by repeating ejection and stop. However, what is essential is that the flow rate or the flow velocity of the liquid is varied, so that the intermittent ejection is not necessarily required.

As illustrated, the actuator 130 includes a first case 131, a second case 132, a third case 133, a piezoelectric element 135, a reinforcing plate 136, and a diaphragm 137. The first case 131 is a cylindrical member. One end of the first case 131 is joined with the second case 32. The other end of the first case 131 is sealed by the third case 133. The piezoelectric element 135 is disposed in a space defined in the interior of the first case 131.

The piezoelectric element 135 is a multi-layer piezoelectric element. One of end portions of the piezoelectric element 135 is fixed to the diaphragm 137 via the reinforcing plate 136. The other end portion of the piezoelectric element 135 is fixed to the third case 133. The diaphragm 137 is formed of a metallic thin film, and is fixed to the first case 131 at a peripheral edge portion thereof. A storage chamber 138 is formed between the diaphragm 137 and the second case 132. The capacity of the storage chamber 138 is changed by driving the piezoelectric element 135.

A first flow channel 139 configured to allow liquid to flow into the storage chamber 138 is formed in the second case 132. The first flow channel 139 is connected to the liquid supply flow channel 140. A second flow channel 134 configured to allow the liquid accommodated in the storage chamber 138 to flow out is formed in the second case 132. The second flow channel 134 is connected to the liquid ejection tube 110.

A drive signal having a predetermined frequency is applied to the piezoelectric element 135 from the control unit 30. The piezoelectric element 135 vibrates at the predetermined frequency upon the reception of the drive signal from the control unit 30. When the piezoelectric element 135 vibrates, the capacity of the storage chamber 138 is changed via the diaphragm 137, and the liquid accommodated in the storage chamber 138 is pressurized. The liquid pressurized or depressurized at the predetermined frequency is provided with pulsation. The liquid passes through the second flow channel 134 and the liquid ejection tube 110 and is ejected toward the outside as pulsed liquid.

The outer tube 120 has an elongated cylindrical shape, and the liquid ejection tube 110 is inserted into the outer tube 120 so as to be slidable in a longitudinal direction in the inside thereof. The length of the outer tube 120 is shorter than the length of the liquid ejection tube 110, and both ends of the liquid ejection tube 110 project from both ends of the outer tube 120 respectively. The outer tube 120 is arranged along part of an inner wall surface of the outer case 50, and is secured to the part of the inner wall surface with an adhesive agent 54.

The slider 62 is coupled to the liquid ejection tube 110. The slider 62 has an ON side on the front side, and an OFF side on the rear side. Therefore, if the slider 62 is slid to ON, the liquid ejection tube 110 moves in a sliding direction that is, to the front side of the handpiece 20. The outer tube 120 is secured to the inner wall surface of the outer case 50 as described above. Therefore, the outer tube 120 does not move, and the liquid ejection tube 110 moves while sliding inside the outer tube 120. A function of the outer tube 120 will be described later.

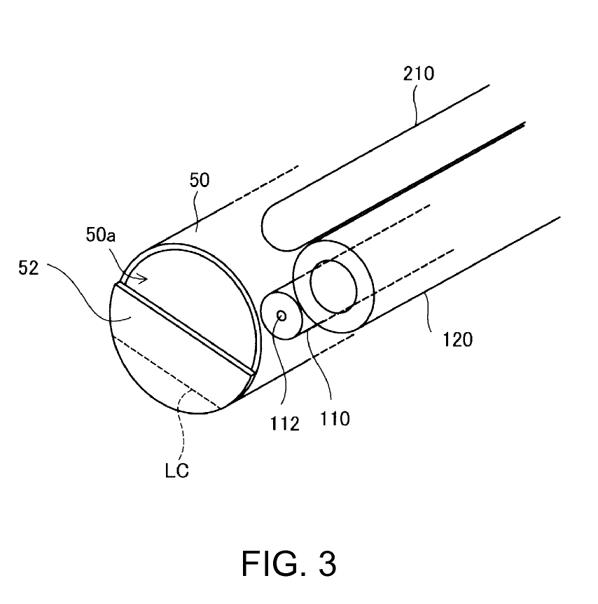

FIG. 3 is a perspective view of a portion in the vicinity of the opening 50a of the outer case 50 illustrating together with a distal end portion of the liquid ejection surgical knife 100 and a distal end portion of the electric surgical knife 200. As illustrated in FIG. 3, a lid member 52 is arranged at a lower half of the opening 50a of the outer case 50. The lower half is located at a position opposing the distal end (end portion on the front side) of the liquid ejection tube 110. The lid member 52 is formed of an elastic material such as silicone rubber, and part of the lid member 52 is adhered to an end surface of the opening 50a with an adhesive agent. If the liquid ejection tube 110 is moved in the sliding direction, that is, to the front side of the handpiece 20, the lid member 52 is bent at a position near a broken line LC (see FIG. 1), and the distal end portion of the liquid ejection tube 110 projects from the outer case 50. The amount of projection in this embodiment is a predetermined value within a range from 1.0 mm to 3.0 mm, and is, for example, 2.0 mm. The actuator 130 is fixed to the handpiece 20. Therefore, even though the slider 62 is moved, the actuator 130 does not move.

The liquid ejection tube 110 has a length sufficient for moving (being deformed) by a sliding movement of the slider 62. The liquid ejection tube 110 is formed of a member having flexibility, and when the slider 62 is OFF, the liquid ejection tube 110 is accommodated in the outer case 50 in a state of being deflected. Reference sign CU in FIG. 2 is a deflecting portion. When the slider 62 is ON, the deflecting portion CU expands and the distal end portion of the liquid ejection tube 110 pushes and bends the lid member 52 and projects from the outer case 50 as described above. In this embodiment, the liquid ejection tube 110 is formed of a PEEK member. The liquid ejection tube 110 may be replaced by various types of flexible members such as polyvinyl chloride, silicon, and thermoplastic elastomer. The liquid ejection tube 110 may be formed of a member having elasticity.

The electric surgical knife 200 includes a high-frequency treatment electrode 210, a first electric cable 220, an actuator 230, and a second electric cable 240.

The first electric cable 220 is an electric line connecting the high-frequency treatment electrode 210 and the actuator 230. The second electric cable 240 is a cable configured to supply power to the actuator 230. The second electric cable 240 is connected to a high-frequency current generator (not illustrated) on the outside of the handpiece 20. The actuator 230 supplies a high-frequency current supplied from the second electric cable 240 to the high-frequency treatment electrode 210 via the first electric cable 220.

The high-frequency current supplied to the high-frequency treatment electrode 210 flows to an affected area. At this time, a Joule heat is generated by a load or a contact resistance, which solidifies protein at the affected area to enable arrest of bleeding.

The actuator 230 includes a switching element configured to receive a control signal from the control unit 30 and turn the high-frequency current to the high-frequency treatment electrode 210 ON and OFF. The actuator 230 performs other various types of control required for the operation of the electric surgical knife 200. The switching element may be provided in the high-frequency current generator. Alternatively, the high-frequency current generator may be accommodated in the actuator 230.

The slider 64 is coupled to the first electric cable 220. The slider 64 has an ON side on the front side, and an OFF side on the rear side. Therefore, if the slider 64 is slid to ON, the first electric cable 220 moves in the sliding direction, that is, to the front side of the handpiece 20. With this movement, the high-frequency treatment electrode 210 moves to the front side, and a distal end of the high-frequency treatment electrode 210 projects from the opening 50a of the outer case 50. The amount of projection in this embodiment is a predetermined value within a range from 1.0 mm to 3.0 mm, and is, for example, 2.0 mm. The actuator 230 is fixed to the handpiece 20. Therefore, even though the slider 64 is moved, the actuator 230 does not move.

The first electric cable 220 has flexibility and a length sufficient for moving by the sliding movement of the slider 64. When the slider 64 is OFF, the first electric cable 220 is accommodated in the handpiece 20 in a state of being deflected.

As illustrated in FIG. 2 and FIG. 3, the liquid ejection tube 110 of the liquid ejection surgical knife 100 and the high-frequency treatment electrode 210 of the electric surgical knife 200 are provided so that the longitudinal directions thereof extend in parallel to each other. A plurality of ejection openings 114 are formed at positions opposing the high-frequency treatment electrode 210 on a side surface of the liquid ejection tube 110. In other words, the plurality of ejection openings 114 are arranged in a line in the longitudinal direction of the liquid ejection tube 110, and oppose the high-frequency treatment electrode 210. The plurality of ejection openings 114 are used for cleaning the high-frequency treatment electrode 210, and the liquid ejection surgical knife 100 provided on the operation instrument 10 of this embodiment operates in a cleaning mode in which the high-frequency treatment electrode 210 is cleaned in addition to a therapy mode in which incision, excision, and arrest of bleeding in the affected area descried above are performed. The cleaning mode will be described in detail below. The therapy mode corresponds to the first mode descried in SUMMARY and the cleaning mode corresponds to the second mode described in SUMMARY.

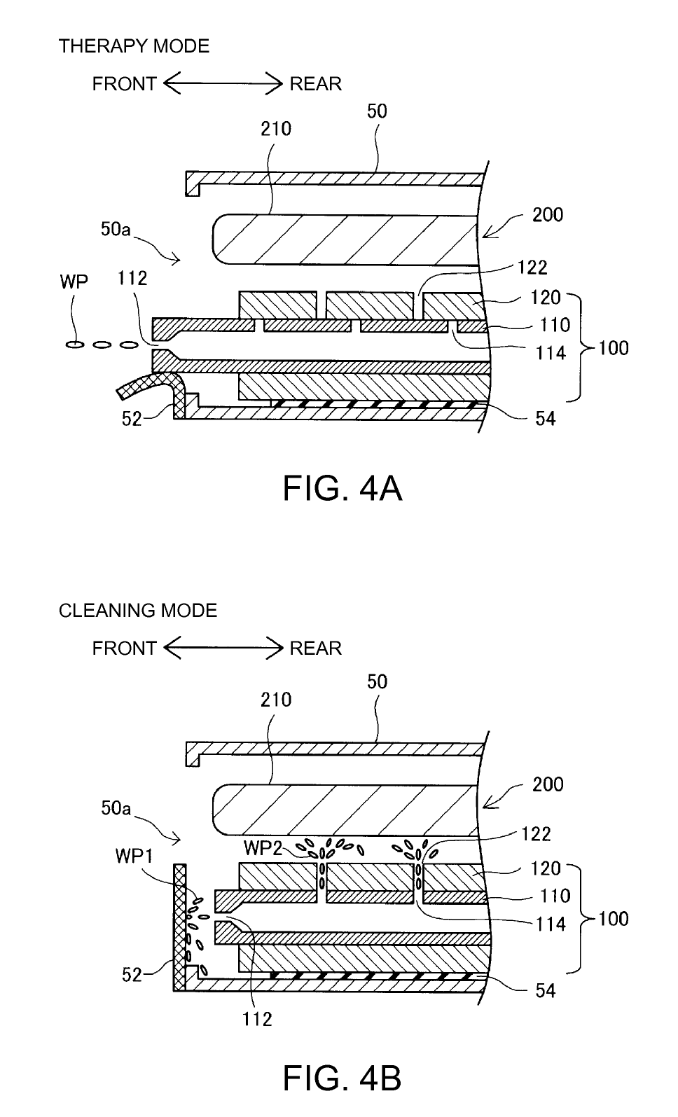

FIGS. 4A and 4B are explanatory drawings for comparing operations of the liquid ejection surgical knife 100 in the therapy mode and in the cleaning mode. As illustrated in FIG. 4A, in the therapy mode, the distal end portion of the liquid ejection tube 110 pushes and bends the lid member 52 and projects from the outer case 50 and, if the foot switch 40 is turned ON in this projecting state, the liquid ejection surgical knife 100 is driven, whereby pulsed liquid WP is ejected from the ejection opening (hereinafter, referred to also as “distal end ejection opening”) 112 formed at the distal end portion. At this time, the plurality of ejection openings (hereinafter, referred to also as “side surface ejection openings”) 114 formed on the side surface of the liquid ejection tube 110 are shielded by an inner wall surface of the outer tube 120, and hence the liquid cannot be ejected therefrom.

In contrast, as illustrated in FIG. 4B, in the cleaning mode, the distal end portion of the liquid ejection tube 110 is retracted into the inside of the outer case 50 and thus accommodated in the outer case 50. If the liquid ejection surgical knife 100 is driven in this state, pulsed liquids WP1 and WP2 are ejected from the distal end ejection opening 112 and the plurality of side surface ejection openings 114, respectively. A plurality of hole portions 122 are provided in the outer tube 120 through which the liquid ejection tube 110 is to be inserted. The number of the plurality of hole portions 122 is the same as the number of the plurality of side surface ejection openings 114. The hole portions 122 and the side surface ejection openings 114 are arranged at the same intervals, and when the liquid ejection tube 110 is at the accommodated position, the plurality of side surface ejection openings 114 and the plurality of hole portions 122 communicate each other. Therefore, the liquid WP2 ejected from the side surface ejection openings 114 is ejected toward the high-frequency treatment electrode 210 opposing the side surface ejection openings 114 through the hole portions 122. Accordingly, the high-frequency treatment electrode 210 of the electric surgical knife 200 can be cleaned with the liquid.

As illustrated in FIGS. 4B and 4A, the liquid ejection tube 110 is movable between a position accommodated in the outer case 50 and a position projecting from the outer case 50. This movement occurs by turning the slider 62 for the liquid ejection surgical knife OFF and ON as has been described thus far, and the relative position of the outer tube 120 with respect to the liquid ejection tube 110 is moved. In the cleaning mode illustrated in FIG. 4B, the respective hole portions 122 provided in the outer tube 120 communicate with the side surface ejection openings 114 provided in the liquid ejection tube 110, whereby the liquid can be ejected from the side surface of the liquid ejection tube 110. In contrast, in the therapy mode illustrated in FIG. 4A, the respective hole portions 122 are located at shifted positions, not communicating with the respective side surface ejection openings 114. Accordingly, the ejection openings 114 are shielded by the inner wall surface of the outer tube 120.

In the cleaning mode in FIG. 4B, the distal end portion of the liquid ejection tube 110 is retracted inward of the outer case 50, and hence the lid member 52 formed of the elastic material is restored from the bent state illustrated in FIG. 4A, and is in a state of closing the lower half of the opening 50a of the outer case 50. Therefore, the liquid WP1 ejected from the distal end ejection opening 112 is shielded by the lid member 52, and is prevented from leaking outside of the outer case 50.

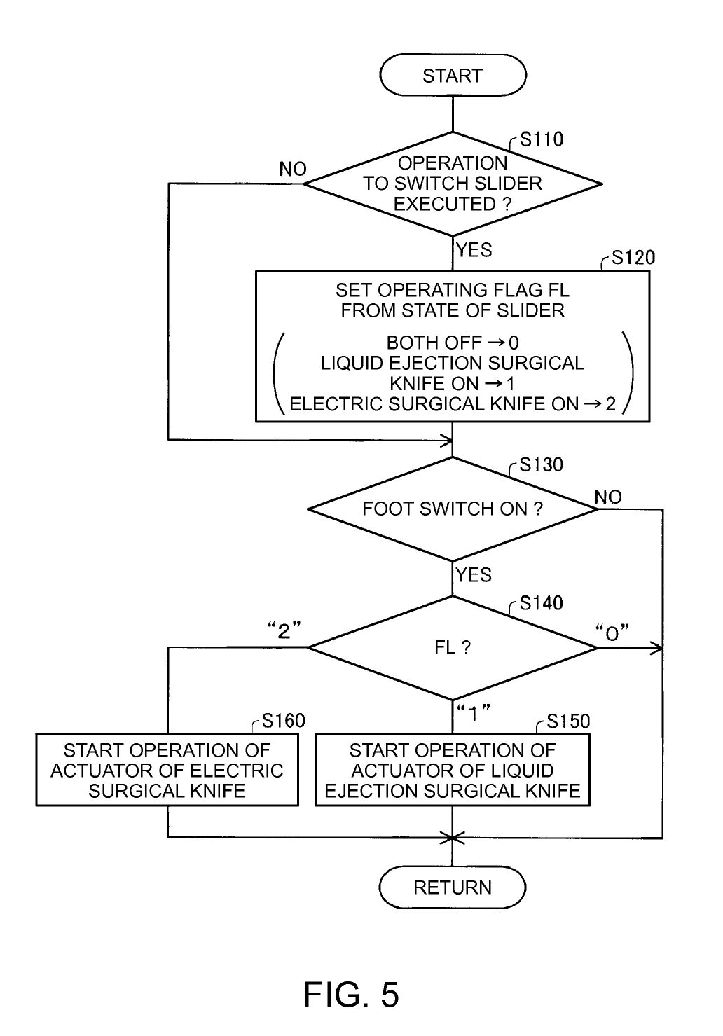

FIG. 5 is a flowchart illustrating a drive routine of the liquid ejection surgical knife 100 and the electric surgical knife 200. The drive routine is repeatedly executed at every predetermined time by the control unit 30. When the process is started, the control unit 30 determines whether or not the operation to switch the sliders 62 and 64 between ON and OFF is performed (Step S110). If it is determined that the switching operating has been performed, a surgical knife selecting flag FL is set from the ON and OFF state of the both sliders 62 and 64 after the switching operation (Step S120). The surgical knife selecting flag FL is a flag indicating the surgical knife to be used. In Step S120, specifically, if both of the sliders 62 and 64 are OFF, a value 0 is set to the surgical knife selecting flag FL. If the slider 62 for the liquid ejection surgical knife is ON, a value 1 is set to the surgical knife selecting flag FL. If the slider 62 for the electric surgical knife is ON, a value 2 is set to the surgical knife selecting flag FL.

After the execution of Step S120, the control unit 30 proceeds the process to Step S130. In contrast, if it is determined that the operation to switch the sliders 62 and 64 is not performed in Step S110, the process in Step S120 is not performed, and the procedure goes to Step S130. In Step S130, the control unit 30 determines whether or not the foot switch 40 is turned ON. Here, if the foot switch 40 is turned ON, the value of the surgical knife selecting flag FL is determined (Step S140), and if the surgical knife selecting flag FL is the value 1, the operation of the actuator 130 of the liquid ejection surgical knife 100 is started (Step S150), and if the surgical knife selecting flag FL is the value 2, the operation of the actuator 230 of the electric surgical knife 200 is started (Step S160). If it is determined that the surgical knife selecting flag FL is the value 0 in Step S140, neither the liquid ejection surgical knife 100 nor the electric surgical knife 200 is operated, and the drive routine is terminated once.

If the actuator 130 of the liquid ejection surgical knife 100 is operated in Step S150, the slider for the liquid ejection surgical knife is switched to ON, and the distal end portion of the liquid ejection tube 110 is at a position projecting from the outer case 50, so that the operation is performed in the therapy mode illustrated in FIG. 4A. Although not written in the illustrated drive routine, if the foot switch is switched from ON to OFF, the actuator started to be operated in Step S150 or S160 is stopped.

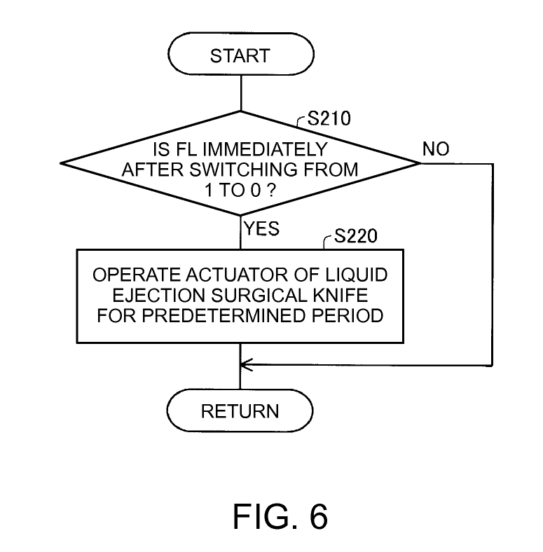

FIG. 6 is a flowchart illustrating an electric surgical knife cleaning routine. The electric surgical knife cleaning routine is processed by the control unit 30 in time sharing with the drive routine in FIG. 5. When the process is started, the control unit 30 determines whether the surgical knife selecting flag FL is switched from the value 1 to the value 0 immediately before, that is, whether or not the slider for the liquid ejection surgical knife is switched from ON to OFF immediately before (Step S210). If it is determined that the switching is performed immediately before, the control unit 30 performs a process of operating the actuator 130 of the liquid ejection surgical knife 100 for a predetermined time (for example, 1 to 3 seconds (Step S220)).

The process in Step S220 is executed irrespective of ON or OFF of the foot switch. When executing the process in Step S220, the slider for the liquid ejection surgical knife is switched to OFF, and the liquid ejection tube 110 is at the position accommodated in the outer case 50, so that the operation is performed in the cleaning mode illustrated in FIG. 4B. After the execution of the process in Step S220, or if it is determined not to be immediately after the switching to OFF in Step S210, the procedure goes to “return”, and the electric surgical knife cleaning routine is terminated.

According to the operation instrument 10 configured as described thus far, the liquid may be ejected to a living tissue in the therapy mode, and the liquid may be ejected toward the high-frequency treatment electrode 210 of the electric surgical knife 200 in the cleaning mode. Therefore, cleaning of the high-frequency treatment electrode 210 of the electric surgical knife 200 is easily achieved in the cleaning mode. Therefore, an operator's labor for cleaning of the electric surgical knife 200 may be reduced, and hence switching between the electric surgical knife 200 and the liquid ejection surgical knife 100 is freely performed. Consequently, the operation instrument 10 of this mode achieves an advantage that efficiency of operations is sufficiently improved. In particular, according to the operation instrument 10 of this embodiment, since the mode is translated to the cleaning mode automatically when the slider for the liquid ejection surgical knife is switched from ON to OFF, the cleaning of the high-frequency treatment electrode 210 is always finished when using the electric surgical knife 200, so that the performance of the electric surgical knife is not lowered. The ejection of liquid to the high-frequency treatment electrode 210 is the intermittent ejection in the same manner as the ejection to the living tissue in the therapy mode. Therefore, the operation instrument 10 of this embodiment is superior in terms of detergency with respect to the high-frequency treatment electrode 210. In this embodiment, the intermittent ejection is employed. However, the invention is not limited to the intermittent ejection, and a continuous ejection is also applicable.

### B. Modifications

The invention is not limited to the embodiment described above, and various modes may be implemented without departing the scope of the invention. For example, the following modifications may be applied.

### Modification 1

In the embodiment described above, liquid is used as fluid to be ejected from the operation instrument 10. In contrast, in this modification, gas may be used as the fluid to be ejected from the operation instrument 10.

### Modification 2

In the embodiment described above, the ejection of the liquid to the high-frequency treatment electrode 210 of the electric surgical knife 200 is performed by providing the plurality of ejection openings 114 in the side surface of the liquid ejection tube 110, and opening and closing these ejection openings 114 by the outer tube 120. In contrast, in this modification, a configuration in which the ejection of the liquid to the electrode portion is performed by providing the ejection opening only at the distal end portion of the liquid ejection tube, pulling the distal end portion with a wire or the like, and bending the ejection opening so as to face the electrode portion of the electric surgical knife is also applicable. Alternatively, the liquid may be ejected to the high-frequency treatment electrode 210 of the electric surgical knife 200 by a branch tube coupled to a generally available liquid ejection tube having the ejection opening only at the distal end portion thereof. The fluid ejection surgical knife may have any configuration as long as the fluid may be ejected toward the electrode portion of the electric surgical knife.

### Modification 3

In the embodiment described above, the side surface ejection openings 114 are opened and closed by shifting the relative position of the liquid ejection tube 110 with respect to the outer tube 120 by moving the liquid ejection tube 110 in a fore-and-aft direction. In contrast, in this modification, a configuration in which the relative position between the outer tube 120 and the liquid ejection tube 110 is shifted by moving the outer tube 120 in the fore-and-aft direction with the liquid ejection tube 110 kept fixed is also applicable. A configuration in which the relative position between the outer tube 120 and the liquid ejection tube 110 is shifted by moving both tubes is also applicable. In addition, a configuration in which the liquid ejection tube 110 is rotated in an axial direction instead of moving in the fore-and-aft direction is applicable, and a configuration in which the outer tube 120 is rotated in the axial direction instead of moving in the fore-and-aft direction is also applicable. Point is that any configuration is applicable as long as the side surface ejection openings 114 may be opened and closed by moving the relative position between the liquid ejection tube 110 and the outer tube 120.

### Modification 4

In the embodiment described above, the high-frequency treatment electrode 210 of the electric surgical knife 200 is kept fixed at the time of cleaning in the cleaning mode. In contrast, in this modification, a configuration in which the high-frequency treatment electrode 210 is moved in the fore-and-aft direction at the time of cleaning in the cleaning mode is also applicable. Accordingly, a range where the liquid comes into contact with the high-frequency treatment electrode 210 may be increased, so that detergency is improved. A configuration in which detergency may be further improved by rotating the high-frequency treatment electrode 210 of the electric surgical knife 200 at the time of cleaning is also applicable.

### Modification 5

In the embodiment described above, when the slider 62 for the liquid ejection surgical knife is switched from ON to OFF, the mode is translated to the cleaning mode. In contrast, in this modification, a configuration in which the mode is translated to the cleaning mode when the slider 64 for the electric surgical knife is switched from OFF to ON, and the distal end portion of the electric surgical knife 200 is projected from the opening 50a of the outer case 50 after the termination of the cleaning mode after having elapsed a predetermined period is applicable.

### Modification 6

In the embodiment described above, as a pulse generating unit of the fluid ejection surgical knife, a mechanism for generating pulsation in the liquid in the storage chamber 138 by the piezoelectric element 135 is employed. In contrast, a mechanism in which air bubbles are generated by irradiating a laser to the liquid, and pulsation is generated in the liquid in the storage chamber by the air bubbles may also be employed. In this case, an optical fiber cable for irradiating the laser in the storage chamber may be connected. The fluid ejection surgical knife may have a mechanism configured to heat the liquid in the storage chamber with an electric heater to generate the air bubbles and provide the fluid with pulsation thereby.

### Modification 7

In the embodiment described above, in the therapy mode and the cleaning mode, a drive voltage of the piezoelectric element or the flow rate of liquid pumped from the pump and a cycle of a pulsed flow may be changed. In the cleaning mode, the drive voltage for driving the piezoelectric element may be set to be larger than the normal drive voltage in the therapy mode. In this configuration, a strong liquid pulse suitable for cleaning may be ejected to achieve efficient cleaning. In the cleaning mode, since the strong liquid pulse suitable for cleaning is also ejected from the plurality of side surface ejection openings, the flow rate of the liquid pumped by the pump may be set to be larger than the flow rate of the liquid pumped from the pump in the therapy mode. In this configuration, the strong liquid pulse suitable for cleaning may be ejected efficiently also from the plurality of side surface ejection openings to achieve efficient cleaning. In the cleaning mode, as described above, by achieving both of setting the drive voltage for driving the piezoelectric element to be larger than the normal drive voltage in the therapy mode and setting the flow rate of the liquid pumped from the pump to be larger than the flow rate in the therapy mode, the advantages of the respective achievements may be obtained.

### Modification 8

In the embodiment described above, cross-sectional areas of the distal end ejection opening and the side surface ejection openings may be differentiated. By setting the side surface ejection openings to be larger than the distal end ejection opening, the amount to be ejected from the side surface may be set to be larger than the amount to be ejected from the distal end, the liquid pulse may be applied to the electric surgical knife efficiently.

### Modification 9

In the embodiment, part of a function realized by software may be realized by hardware, or part of the function realized by hardware may be realized by software.

The invention is not limited to the embodiment, examples, and modifications described above, and may be implemented in various configurations without departing the scope of the invention. For example, technical characteristics in the embodiment, the examples, and the modifications corresponding to the technical characteristics in the respective modes described in the paragraph of the summary may be replaced or combined as needed in order to solve part or entire part of problems described above or in order to achieve part or entire part of the above-described advantages. The technical characteristics may be eliminated as needed unless otherwise specified to be essential.

The invention may be configured to be capable of discharging solid substances, semi-solid substances, or liquid generated by cleaning the electric surgical knife by a sucking source and a sucking route configured to communicate therewith, not illustrated, safely to the outside of the body irrespective of the embodiments, examples, or modifications.

What is claimed is:

1. An operation instrument comprising:
an electric surgical knife;
a fluid ejection surgical knife capable of ejecting fluid; and
an outer case configured to accommodate the electric surgical knife and the fluid ejection surgical knife, and including an opening which allows an electrode portion of the electric surgical knife to project therefrom, wherein
the fluid ejection surgical knife is operated so as to be switched between a first mode in which fluid is ejected to the outside of the outer case, and a second mode configured to eject the fluid toward the electrode portion of the electric surgical knife in a state of being accommodated in the outer case.

2. The operation instrument according to claim 1, wherein
the fluid ejection surgical knife includes:
a pulse generating unit configured to generate pulsation in fluid;
an ejection tube communicating with the pulse generating unit; and
an outer tube in which the ejection tube is inserted,
wherein
the ejection tube includes:
a first ejection opening formed at a distal end portion of the ejection tube, and
a second ejection opening formed in the ejection tube at a position opposing the electrode portion of the electric surgical knife,
the outer tube includes:
a hole portion at a position capable of aligning with the second ejection opening, and
the second ejection opening and the hole portion communicate with each other by a relative movement of the ejection tube and the outer tube.

3. The operation instrument according to claim 2, wherein
the fluid ejection surgical knife is configured in such a manner that
the second ejection opening and the hole portion are brought into communication with each other by projecting the distal end portion of the ejection tube from the outer case in the first mode and retracting the ejection tube into the inside of the outer case in the second mode.

4. An operation instrument comprising:
an electric surgical knife;
a fluid ejection surgical knife capable of ejecting fluid; and
an outer case configured to accommodate the electric surgical knife and the fluid ejection surgical knife, and including an opening which allows an electrode portion of the electric surgical knife to project therefrom, wherein
in a fluid passage of the fluid ejection surgical knife is provided with an opening from which the fluid can be ejected toward the electrode portion of the electric surgical knife in a state in which the electric surgical knife and the fluid ejection surgical knife are accommodated in the outer case.
